(19) United States
(12) Patent Application Publication
(10) Pub. No.: US 2015/0282798 A1
(43) Pub. Date: Oct. 8, 2015

(54) Oral Tool

(71) Applicant: Michele Uphoff, Oregon City, OR (US)

(72) Inventor: Michele Uphoff, Oregon City, OR (US)

(21) Appl. No.: 14/676,412

(22) Filed: Apr. 1, 2015

Related U.S. Application Data
(60) Provisional application No. 61/973,909, filed on Apr. 2, 2014.

(51) Int. Cl. A61B 17/02

(52) U.S. Cl. CPC A61B 17/025

(57) ABSTRACT

An oral tool having a series of differently sized spherical members ranging from small to large that can be removably attached to a rod member via a screwing mechanism, wherein the device is adapted to be inserted into one's mouth to be used in sequential order in a gradual manner over time in order to effectuate incremental improvements in one's jaw, mouth, and facial muscles and/or areas to relieve pain and injury thereof. In use, one can initially utilize the smallest sized spherical member and gradually increase to the next increasing size spherical member until the largest sized spherical member can be utilized. The user can employ a sucking motion with one's mouth once the device is inserted into one's mouth in order to relieve pain and/or muscle stiffness in one's mouth, jaw and/or facial muscles due to oral surgery and temporomandibular joint disorders.

### CROSS REFERENCE TO RELATED APPLICATION

This application claims the benefit of U.S. Provisional Application No. 61/973,909 filed on Apr. 2, 2014. The above identified patent application is herein incorporated by reference in its entirety to provide continuity of disclosure.

### BACKGROUND OF THE INVENTION

1. Field of the Invention

The present invention relates to an oral aid that can be placed within the mouth of the user in order to supplement and/or to be used in conjunction with oral and/or dental treatment. More specifically, the invention comprises several interchangeable spherical members having various sizes to be used in consecutive order in order to gradually expand and stretch one's mouth, jaw, and facial muscles over time.

Temporomandibular joint disorder can cause pain, stiffness, or tenderness in the face, jaw joint area, neck and shoulders in addition to causing pain and discomfort while chewing, speaking, and/or opening one's mouth and moving one's jaw. Further, some individuals may experience lock jaw or a clicking, popping, and/or grating sound in one's jaw when yawning or performing other tasks that require the opening and closing of one's jaw.

Other jaw, mouth, and face related issues involve pain, tenderness and swelling thereof after undergoing oral surgery. This can lead to stiff, painful and injured muscles and tissues and can take weeks or months to heal. Several methods to effectuate healing involve the use of exercises that engage the muscles and joints of the mouth, face, and jaw. However, these can be ineffective as they involve self-actuated movements of the mouth, jaw, and face and do not offer secondary tools to help effectuate these movements.

Accordingly, the present invention relates to a new and improved oral tool that helps to heal and relieve pain, stiffness and injury in the muscles and joints of the mouth, face and jaw of the user. The present invention provides a tool comprising a series of spherical members having increased sizes and a rod member, wherein the user can secure the smallest sized spherical member onto the rod, and then place the spherical member into one's mouth and employ a sucking motion for a period of time. The user can then utilize a slightly larger spherical member and place that spherical member into his or her mouth for a period of time. The user can proceed in this fashion, utilizing spherical members of increasing size in order to gradually effectuate incremental changes and improvement in the joints and muscles of one's mouth, face, and jaw.

2. Description of the Prior Art

Devices have been disclosed in the prior art that relate to oral tools insertable into the mouth of the user. These include devices that have been patented and published in patent application publications. These devices generally relate to oral tools or apparatuses that can be inserted into the mouth of the user to relieve smoking addictions or exercise the muscles of the face, mouth and throat. The following is a list of devices deemed most relevant to the present disclosure, which are herein described for the purposes of highlighting and differentiating the unique aspects of the present invention, and further highlighting the drawbacks existing in the prior art.

Specifically, U.S. Pat. No. 7,083,548 to Moore provides a soft and flexible tool that can be used for conditioning facial muscles to be used for cosmetic and medical purposes. The device can be used via sucking on or compressing the body thereof in order to exercise the muscles in the mouth, face and throat. The present invention however, provides a series of oral tools adapted to be used in a gradual and consecutive fashion in order to expand and stretch one's jaw, facial muscles, and/or mouth.

U.S. Pat. No. 6,458,149 to Peters-Combs discloses a device and method for treating stress-induced habits such as smoking, drinking, over-eating and the like. The device comprises an adult pacifier having various embodiments, wherein the device can be impregnated with a flavor such as cherry, apple, wintergreen and the like. One embodiment can provide a balloon shaped tongue engaging member and can provide a rod member attached thereto. While this invention may be helpful in the way of breaking habits regarding mouth fixations, it offers little utility in the way of expanding one's mouth and stretching one's facial muscles or jaw.

U.S. Pat. No. 8,105,210 to Seybold describes a jaw relaxation exercise appliance and method of use that can be utilized to relieve the symptoms associated with the temporal mandibular joint. The device can be placed in the mouth for short intervals during the day, wherein the user can slide one's lower jaw forward and rearward along the device to exercise the muscles thereof. Still, the present invention provides a series of oral tools of varying sizes to be used gradually and over time in order to effectuate incremental improvements in one's jaw, mouth, and facial muscles and/or areas.

U.S. Pat. No. 4,951,691 to Leary provides for a method for treating and curing smoking habits, wherein the device comprises an oval, disc-shaped object adapted to be placed into one's mouth and onto the tongue in order to alleviate the urge to smoke in addition to over eating. The device can be attached to a necklace or chain and worn around the neck of the user to be readily available and accessible. Still, the present invention includes a series of different sized spherical members attached to a rod member to be inserted into the mouth of the user for relieving pain associated with one's jaw, mouth and facial muscles.

U.S. Pat. No. 3,805,771 to Wright provides a facial muscle exerciser that includes a disc that is insertable between the lips and teeth, wherein the disc is connected to a barrel and a plastic handle to which the user can hold and insert into his or her mouth. The device can be used to restore muscle tone and can prevent the formation of wrinkles, wherein the device can exert pressure onto the lips. The present invention can be utilized to exercise the muscles of the face and can further relieve pain and stiffness of the mouth, jaw and facial muscles after oral surgery thereof.

U.S. Pat. No. 4,551,329 to Harris discloses an oral medicament in the form of a lollipop having a handle, wherein the device can be utilized to dispense medication onto the mouth of the user. The present invention however, comprises a series of spherical members to be used in a gradual manner, wherein the user increases the size of the spherical member to be used in a consecutive manner in order to stretch the muscles of the mouth, jaw and face in order to relieve pain, pressure, and stiffness thereof.

These prior art devices have several known drawbacks. The above discussed oral devices do not provide for an oral tool that provides a series of different sized spherical structures that can be removably attached to a rod member via a screwing mechanism, wherein the device is adapted to be inserted into one's mouth to be used in a gradual manner over time in order to effectuate incremental improvements in one's jaw, mouth, and facial muscles and/or areas and to relieve pain thereof. It is submitted that the present invention substantially diverges in design elements from the prior art and consequently it is clear that there is a need in the art for an improvement to existing oral insertion devices. In this regard the instant invention substantially fulfills these needs.

### SUMMARY OF THE INVENTION

In view of the foregoing disadvantages inherent in the known types of oral tools and apparatuses now present in the prior art, the present invention provides a new oral tool wherein the same can be utilized for providing convenience for the user when inserting the oral tool into the mouth over a gradual period of time in order to effectuate incremental changes in one's jaw, mouth, and facial muscles and/or areas as well as expanding and stretching one's jaw, mouth, and facial muscles.

It is therefore an object of the present invention to provide a new and improved oral tool device that has all of the advantages of the prior art and none of the disadvantages thereof.

It is another object of the present invention to provide an oral tool having a series of different sized mouth insertable tools to be used in a gradual fashion over time to effectuate incremental changes to the user's mouth, jaw, and facial muscles and/or areas via stretching and engaging the muscles and joints thereof.

Another object of the present invention is to provide an oral tool having a rod member on which the user can grip in order to place it into one's mouth.

Another object of the present invention is to provide an oral tool having a series of spherical members of varying sizes adapted to expand and/or stretch one's jaw, mouth, and/or facial muscles.

Yet another object of the present invention is to provide an oral tool that can be flavored.

Yet another object of the present invention is to provide an oral tool that can be easily washed and/or cleaned.

Another object of the present invention is to provide an oral tool that can be pliable.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a view of the oral tool having a spherical member being attached to a rod member, wherein the oral tool is being inserted into the mouth of the user.

FIG. 2 shows a view of the oral tool having a spherical member that includes a recessed channel in which the rod member can be inserted therein for attachment thereto via a screwing mechanism.

### DETAILED DESCRIPTION OF THE INVENTION

Reference is made herein to the attached drawings. Like reference numerals are used throughout the drawings to depict like or similar elements of the oral tool. For the purposes of presenting a brief and clear description of the present invention, the preferred embodiment will be discussed as used for inserting the oral tool into one's mouth in order to expand and/or stretch the mouth, jaw and/or facial muscles in order to effectuate an incremental improvement of the described areas above. The figures are intended for representative purposes only and should not be considered to be limiting in any respect.

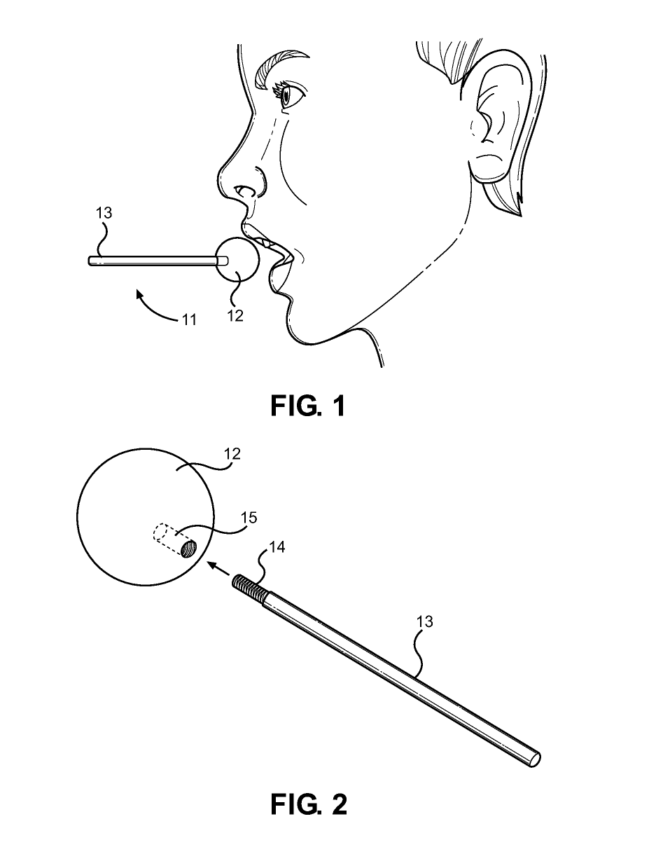

Referring now to FIG. 1, there is shown a view of the oral tool 11 that includes a series of differently sized spherical members, wherein each spherical member 12 includes a recessed channel that extends from the interior of the spherical member and terminates at the exterior surface thereof forming an open end in which a rod member 13 can be inserted. The spherical member 12 provides a spherically shaped structure that is solid or hollow, whereby the hollow embodiment forms an interior volume. The rod member 13 is elongated and includes a first end and a second end, wherein the first end includes a threaded portion and the recessed channel provides a corresponding threaded interior. The user can insert the threaded portion at the first end of the rod member 13 into the open end of the recessed channel and rotate the rod member 31 in order to fasten the threaded portion of the rod member 13 with the threaded interior of the recessed channel of the spherical member 12 for securement thereto, thus forming one unitary body.

The oral tool 11 comprises a plurality of spherical members having different sizes. The plurality of spherical members include diameters that increase at a fixed interval. In one embodiment, there is a first, second, and third spherical member. The first spherical member has a diameter of 2 cm, the second has a diameter of 3 cm and the third has a diameter of 4 cm. The spherical members are intended to be used gradually over a period of time in order to effectuate an incremental improvement in the stretching or flexibility of the mouth, jaw and/or facial muscle and joint areas. In use, the user can manually grasp the rod member 13 having a small spherical member 12 attached thereto for initial use to be inserted into one's mouth in order to expand and/or stretch the facial and/or jaw muscles for a period of time. Once inserted into the mouth, the user can hold the oral tool 11 therein and allow it to rest against the interior cheek, roof of the mouth, lips and the like while rubbing and/or massaging the spherical member 12 against the interior cheek and gums of one's mouth in addition to employing a sucking motion in order to further relieve pain, stiffness and injury in the muscle and joint areas.

The user can continue to engage in the above described action until pain and stiffness has subsided and muscle and joint pliability has increased in the jaw and facial regions. Thereafter, the user can repeat the above described method for a period of time using the next increasing size spherical member 12 and so on and so forth until the user has effectuated an improvement in the jaw, mouth and/or facial muscles and joint regions. The time period in which the user can utilize each of the spherical members 12 can range from a few days, one week, and/or a few weeks. However, in a preferred embodiment, the smallest spherical member 12 should be utilized for a period of thirty days, the next increasing sized spherical member 12 should be utilized for a period of sixty days, and the next increasing size should be utilized for a period of ninety days in order to effectuate an optimal and/or substantial improvement in the flexibility and pain relief in one's facial and/or jaw muscles.

FIG. 2 shows a view of the oral tool 11 having a spherical member 13 that includes a recessed channel 15 in which the rod member 13 can be inserted therein for attachment thereto via a screwing mechanism. The user can place the threaded portion 14 of the rod member 13 into the recessed channel 15 of the spherical member 12 in order to affix the rod member 13 to the spherical member 12. However, the attachment method of the rod member 13 to the spherical member 12 need not be via a screwing mechanism and can include magnetic, adhesive, snap and/or other suitable fasteners. In an alternative embodiment, the oral tool 11 can have the rod member 13 integral with the spherical member 12 thus forming one unitary body, wherein the rod member 13 and the spherical member 12 is permanently affixed to one another.

The exterior of the spherical member 12 or the spherical member 12 in its entirety can be embedded with a flavor, wherein the flavor can be released once the user places the spherical member into his or her mouth. The flavors can include cherry, grape, strawberry, lemon, orange, banana, coconut, peach and/or other suitable flavors. However, the oral tool 11 need not be flavored in an alternative embodiment and can be non-flavored. The spherical member 12 can consist of a hollow structure or can be solid in structure. Further, the spherical member 12 may be slightly flexible, wherein once the user places the spherical member 12 into the mouth of the user, the user can bite down onto the spherical member 12 whereby it provides a slight softness or cushioned texture when biting thereon to further engage the mouth, jaw and facial muscles of the user. The oral tool 11 can be made from food grade plastic, rubber, silicone, medical grade stainless steel, and/or other suitable materials.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. An oral tool comprising:
a rod; said rod having a first end and a second end;
at least one spherical member; said spherical member having an exterior and interior;
wherein said at least one spherical member is secured to said first end of said rod.

2. The oral tool of claim 1, said at least one spherical member is removably secured to said first end of said rod.

3. The oral tool of claim 1, wherein said first end of said rod further comprises threading thereon.

4. The oral tool of claim 1, wherein said at least one spherical member comprises a recessed channel.

5. The oral tool of claims 1 and 3, wherein said at least one spherical member comprises a recessed channel having a threaded interior that extends into the interior thereof having an open end, said open end is flush with said exterior of said at least one spherical member, wherein said threading on said first end of said rod is inserted into said open end of said recessed channel of said at least one spherical member for engagement therewith.

6. The oral tool of claim 1, wherein said at least one spherical member further comprises a series of spherical members of varying sizes, wherein said series of spherical members of varying sizes are adapted to be used in increasing order to effectuate incremental changes in jaw, mouth and facial muscles.

7. The oral tool of claim 1, wherein said spherical member is embedded with a flavor.

8. The oral tool of claim 1, wherein said spherical member is pliable.

9. The oral tool of claim 1, wherein said spherical member is substantially rigid.

10. The oral tool of claim 1, wherein said at least one spherical member is permanently secured to said first end of said rod.
